(19) United States
(12) Patent Application Publication
(10) Pub. No.: US 2007/0092489 A1
(43) Pub. Date: Apr. 26, 2007

(54) Use of receptor sequences for immobilizing gene vectors on surfaces

(75) Inventors: Ilia Fishbein, Philadelphia, PA (US); Ivan Alferiev, Clementon, NJ (US); Robert J. Levy, Merion Station, PA (US); Origene Nyanguile, Auderghem (BE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: THE CHILDREN'S HOSPITAL OF PHILADELPHIA, Philadelphia, PA (US)

(21) Appl. No.: 10/567,872

(22) PCT Filed: Aug. 13, 2004
(86) PCT No.: PCT/US04/26509

§ 371 Date: May 19, 2006

Related U.S. Application Data
(60) Provisional application No. 60/494,886, filed on Aug. 13, 2003.

(51) Int. Cl. A61K 48/00; C12N 15/861

(52) U.S. Cl. 424/93.2; 435/456; 424/423; 977/802

(57) ABSTRACT

The present invention relates to compositions and methods of immobilizing a viral vector to an implantable medical device, for example a vascular stent. Specifically, a composition for delivery of a therapeutic agent is provided which includes: a gene transfer vector, a surface and a modified protein, wherein the gene transfer vector is bound to the modified protein and the modified protein is covalently bound to the surface and wherein the composition is adapted to deliver the gene transfer vector to a mammalian cell. The viral vector is preferably an adenoviral vector and the modified protein is preferably CAR D1.

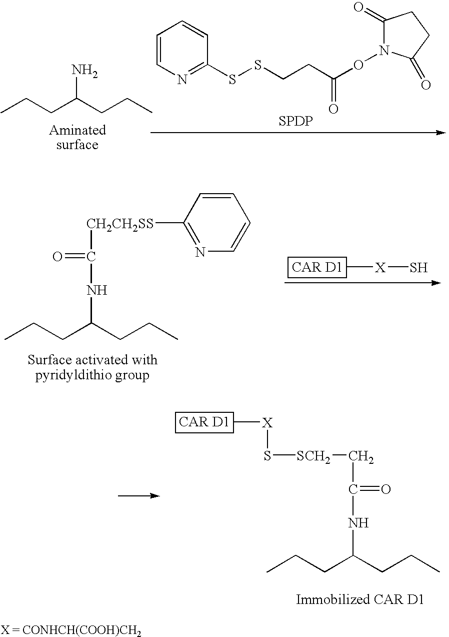

### CROSS-REFERENCE TO RELATED APPLICATIONS

This application claims the benefit of provisional Application No. 60/494,886 filed on Aug. 13, 2003, which is incorporated herein in its entirety.

### STATEMENT REGARDING FEDERALLY SPONSORED RESEARCH OR DEVELOPMENT

This research was supported in part by U.S. Government funds (National Heart, Lung and Blood Institute grant number NHLBI HL72108), and the U.S. Government may therefore have certain rights in the invention.

### BACKGROUND OF THE INVENTION

1. Field of Invention

This invention relates to the preparation of a medical device surface to be used as a viral vector delivery system. It also relates to the use of the coxsackie-adenovirus receptor (CAR) fragment D1 (CAR D1) complexed with other entities to facilitate cell entry.

2. Description of Related Art

There is a need for localized or regional delivery of nucleic acids, such as DNA, for use in the treatment of a variety of diseases by gene therapy and as a preventative or adjunct to other therapeutic modalities. Through gene therapy, it is possible to treat both genetic diseases (e.g. cancer, hemophilia) and infectious diseases (e.g. AIDS) by introducing exogenous genetic material into selected cells. Although tremendous progress has been made in the area of gene therapy, problems still exist regarding the immunogenicity of the exogenous nucleic acid, as well as site-specific cell entry of the vector into a targeted cell. Thus, there is a need for a biologically compatible method of site specific delivery of gene constructs, which may be incorporated and used with traditional implantable medical devices, or may be used with bioresorbable devices. The use of medical devices, such as vascular stents, catheters and the like, has been proposed to deliver nucleic acids that encode proteins or peptides directly related to the function of or recognized effects with medical devices.

The use of recombinant viral vectors for the delivery of exogenous genes to mammalian cells is well established. See e.g. Boulikas, T. in Gene Therapy and Molecular Biology Volume 1, pages 1-172 (Boulikas, Ed.) 1998, Gene Therapy Press, Palo Alto, Calif. However, certain viral vectors commonly used in such instances, such as adenoviruses, exhibit a broad tropism which permits infection and expression of the exogenous gene in a variety of cell types. While this can be useful in some instances, the treatment of certain diseases is enhanced if the virus is able to be modified so as to “target” (e.g., to preferentially infect) only a limited type of cell or tissue.

A variety of approaches to create targeted viruses have been described in the literature. For example, cell targeting has been achieved with adenovirus vectors by selective modification of the viral genome knob and fiber coding sequences to achieve expression of modified knob and fiber domains having specific interaction with unique cell surface receptors. Examples of such modifications are described in Wickham et al. (1997) J. Virol. 71(11):8221-8229 (incorporation of RGD peptides into adenoviral fiber proteins); Arnberg et al. (1997) Virology 227:239-244 (modification of adenoviral fiber genes to achieve tropism to the eye and genital tract); Harris and Lemoine (1996) TIG 12(10):400-405; Stevenson et al. (1997) J. Virol. 71(6):4782-4790; Michael et al. (1995) Gene Therapy 2:660-668 (incorporation of gastrin releasing peptide fragment into adenovirus fiber protein); and Ohno et al. (1997) Nature Biotechnology 15:763-767 (incorporation of Protein A-IgG binding domain into Sindbis virus).

As used herein, the term “gene transfer vector” generally refers to all vectors with which one or more therapeutic genes can be transferred or introduced into the desired target cells and, in particular, viral vectors having this property. In the majority of cases of gene therapy, a viral vector is used to introduce the gene to be expressed into appropriate cells. Gene transfer is most commonly achieved through a cell mediated ex vivo therapy in which cells from the blood or tissue are genetically modified in the laboratory and subsequently returned to the patient. Viral vectors have been widely used in gene transfer due to the relatively high efficiency of transfection and potential long-term effect through the actual integration into the host's genome. Adenoviral vectors, in particular, have a relatively low toxicity to host cells, efficiently infect a broad range of host cells, and do not typically integrate into the host cell genome; therefore, they are among the preferred contemporary gene transfer vectors.

There is, however, a substantial number of cell types that adenoviral vectors do not efficiently infect. Moreover, for some applications, there has been a desire in the art to limit the host cell range of adenoviral vectors. Accordingly, there has been a significant effort to make fusion adenoviral vectors having modified coat proteins, which change and control the efficiency with which adenoviral vectors infect host cells in vivo and in vitro (see, e.g., U.S. Pat. No. 4,593,002 (Dulbecco), U.S. Pat. No. 5,521,291 (Curiel et al.), U.S. Pat. No. 5,543,328 (McClelland et al.), U.S. Pat. No.5,547,932 (Curiel et al.), U.S. Pat. No.5,559,099 (Wickham et al.), U.S. Pat. No. 5,695,991 (Lindholm et al.), U.S. Pat. No. 5,712,136 (Wickham et al.), and International Patent Application WO 94/10323 (Spooner et al.)). These modified coat proteins bind or selectively bind to a protein on the surface of a cell, which mediates the uptake of the receptor.

Earlier studies have also utilized anti-vector antibodies to surface immobilize adenoviral gene vectors to medical devices in order to facilitate vector delivery. Avidin-biotin affinity has been used as well to immobilize adenoviral vectors. However, avidin is highly immunogenic which represents a major limitation for any consideration related to human use.

Thus, there exists a need in the art for an adenoviral gene transfer vector or a method for producing an adenoviral vector that can facilitate cell entry, which also allows easy and efficient vector production and whose means of immobilization does not elicit an immune response. The present invention provides compositions and methods for utilizing a human recombinant protein to immobilize a viral vector to a surface, preferably a medical device, and to target said viral vector to a particular cell. Compared to prior art viral vectors, the compositions described herein exhibit reduced immunogenicity and enhanced delivery of the viral vector to desired cells.

The present invention also includes methods that use intein-mediated protein ligation (IPL) to fuse a non-immunogenic adenoviral receptor protein or peptide to a receptor targeting ligand (e.g., cellular ligand) which can then bind a specific cell type. Thus, the present invention provides a convenient method of generating functional biological molecules that mediate adenovirus targeting to specific cells, for example cancer cells.

These and other advantages of the present invention, as well as additional inventive features, will be apparent from the description of the invention provided herein.

All references cited herein are incorporated by reference in their entireties.

### BRIEF SUMMARY OF THE INVENTION

In one aspect, the invention includes a composition comprising a surface and a modified protein, and optionally a gene transfer vector, wherein the gene transfer vector is bound to the modified protein and the modified protein is covalently bound to the surface. In one embodiment, the gene transfer vector is adapted to bind to a receptor on the mammalian cell and wherein the modified protein comprises at least one of a fusion protein and a polypeptide. In another embodiment, the modified protein is covalently bound to the surface through a thiol residue and a linker. In a further embodiment, the gene transfer vector is a viral vector. In a preferred embodiment, the viral vector is an adenovirus vector. In a more preferred embodiment, the adenovirus vector is a member selected from the group consisting of a first-generation adenovirus vector, a second-generation adenovirus vector, an adenovirus vector of large DNA capacity and a deleted adenovirus vector.

In another embodiment, the surface is a metal surface. In a preferred embodiment, the metal surface is a surface of a medical device and the medical device is selected from the group consisting of a stent, a heart valve, a wire suture, a joint replacement, a urinary dilator, an orthopedic dilator, a catheter and a endotracheal tube. In one embodiment, the medical device is at least one of an internal device and an external device. In another embodiment, the medical device is coated with a layer of the linker, a layer of the modified protein and a layer of the gene transfer vector.

In another embodiment, the fusion protein is generated through intein-mediated protein ligation. In a further embodiment, the fusion protein comprises at least a fragment of a CAR protein and a receptor targeting ligand. In a preferred embodiment, the fragment of the CAR protein is an extracellular domain of CAR or an immunoglobulin D1 domain of CAR. In another preferred embodiment, the receptor targeting ligand is selected from the group consisting of apolipoprotein E, transferrin, a vascular endothelial growth factor, a transforming growth factor-beta, a fibroblast growth factor, an RGD containing peptide, folic acid or virtually any ligand-receptor pair entity. In another preferred embodiment, the receptor is selected from the group consisting of a lipoprotein receptor, a transferrin receptor, a VEGF receptor, a TGF-beta receptor, an FGF receptor, a recombinant integrin receptor protein, a folic acid receptor, a folate receptor or virtually any ligand receptor pair entity.

In another aspect, the invention includes a method for preparing the composition of the invention, the method comprising: (a) providing a protein; (b) modifying the protein with a reagent to contain a reactive group, thereby yielding a modified protein; (c) providing a surface; (d) treating the surface with a surface modifier comprising a linker and a functional group; (e) reacting the modified protein with the functional group on the surface in order to covalently bind the modified protein to the surface via the linker; and optionally (f) binding the gene transfer vector to the modified protein. In one embodiment, the protein is a CAR protein or fragment of CAR. In another embodiment, the fragment of CAR is an immunoglobulin D1 domain of CAR. In a further embodiment, the protein is a fusion protein. In another embodiment, the fusion protein comprises a fragment of CAR ligated to a receptor targeting ligand by intein-mediated protein ligation. In a preferred embodiment, the fragment of CAR is an extracellular domain of CAR or an immunoglobulin D1 domain of CAR. In another preferred embodiment, the receptor targeting ligand is selected from the group consisting of apolipoprotein E, transferrin, a vascular endothelial growth factor, a transforming growth factor-beta, a fibroblast growth factor, an RGD containing peptide and folic acid.

In one embodiment, the reagent is a cysteine and the reactive group is a thiol group or an avidin-biotin affinity construct. In another embodiment, the surface is a surface of a medical device and the medical device is selected from the group consisting of a stent, a heart valve, a wire suture, a joint replacement, a urinary dilator, an orthopedic dilator, a catheter and a endotracheal tube. In one embodiment, the medical device is at least one of an internal device and an external device.

In another embodiment, the surface modifier is polyallylamine bisphosphonate, the linker is an entity containing a reactive succinimide and a pyridyl-dithiol group, and the functional group is selected from the group consisting of an amino group, a sulfhydryl group, biotin reactive succinimides, epoxy-residues and aldehyde functionalities. In a further embodiment, the gene transfer vector is a viral vector. In a preferred embodiment, the viral vector is an adenovirus vector and the adenovirus vector is a member selected from the group consisting of first-generation adenovirus vector, second-generation adenovirus vector, adenovirus vector of large DNA capacity and deleted adenovirus vector.

In another aspect, the invention includes a method of delivering a viral vector to an animal tissue, the method comprising administering to a body location in fluid communication with the animal tissue the composition of the invention.

### BRIEF DESCRIPTION OF SEVERAL VIEWS OF THE DRAWINGS

The invention will be described in conjunction with the following drawings in which like reference numerals designate like elements and wherein:

FIG. 1A is a schematic representing a process of making a thiolated CAR D1 protein.

FIG. 1B is a schematic representing the utilization of a surface modifier to immobilize the activated CAR D1 to a surface.

FIG. 2 is a schematic representing the synthesis of adenoviral targeting molecules.

FIG. 3A is a graph representing the number of green fluorescent protein (GFP) positive cells after lipoprotein related receptor (LRP)-mediated Ad transduction of the primary human fibroblast HDF cell line.

FIG. 3B is a graph representing the targeting ability of the D1-apoE fusion protein.

FIG. 4A is a graph representing the number of GFP positive cells after fibroblast growth factor receptors (FGFRs)-mediated Ad transduction of ovarian adenocarcinoma SKOV-3 cells.

FIG. 4B is a graph representing the targeting ability of the D1-fibroblast growth factor 2 (FGF2) fusion protein.

FIG. 5A is a graph representing the number of GFP positive cells after folate receptors (FRs)-mediated Ad transduction of KB cells (ATCC CCL-17).

FIG. 5B is a graph representing the targeting ability of the D1-folate fusion protein.

FIG. 6 is a table representing the correlation between binding affinity and amount of CAR D1 targeting molecules required for optimal targeted gene delivery.

### DETAILED DESCRIPTION OF THE INVENTION

The present invention provides compositions in which a gene transfer vector or other delivery vehicle is attached by a coordinate covalent linkage to a targeting ligand. Such delivery vehicles include, in addition to viral vectors, other molecules or carriers that are capable of delivering an agent to a cell. Liposomes, for example can be engineered to accept the coordinate covalently linked targeting ligands, as can molecules that bind to nucleic acids or other agents.

In a preferred embodiment, the gene transfer vector is a viral vector to which targeting ligands are attached. The term “virus” is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. The viruses possess virally encoded viral coat proteins. The viruses useful in the practice of the present invention include recombinantly modified enveloped or non-enveloped DNA and RNA viruses. In presently preferred embodiments, the viruses are selected from baculoviridiae, parvoviridiae, picornoviridiae, herpesviridiae, poxviridae, or adenoviridiae. Fusion viral vectors which exploit advantageous elements of each of the parent vector properties (See e.g., Feng et al. (1997) Nature Biotechnology 15:866-870) can also be employed in the practice of the present invention.

This invention relates to the use of a human recombinant protein to tether a gene transfer vector to a surface in order to introduce exogenous nucleic acid into a cell without eliciting an immune response. In addition, this recombinant protein-gene transfer vector complex interacts with other proteins on the cell surface to facilitate cell entry, thus enhancing expression of the transgene generated by the gene transfer vector. In a preferred embodiment, the human recombinant protein is the D1 fragment of the coxsackie-adenovirus receptor (hereinafter referred to as CAR D1). In another preferred embodiment, the gene transfer vector is an adenoviral vector.

Adenoviruses are a relatively homogeneous group of viruses characterized by an icosahedral capsid, which consists mainly of the virally encoded hexon, penton and fiber proteins, and of a linear, double-stranded DNA genome with a size of about 36 kilobases (kb). At its ends, the viral genome contains the inverted terminal repeat sequences (ITRs), which comprise the viral origin of replication. At the left-hand end of the genome there is a packaging signal, which is necessary for packaging of the viral genome into the virus capsids during an infection cycle. There are more than 40 different human serotypes based on parameters that discriminate between the various serotypes, such as hemagglutination, tumorigenicity and DNA sequence homology (Wigand et al., in: Adenovirus DNA, Doerfler ed., Martinus Nijoff Publishing, Boston, pp. 408-441, 1986). Adenoviral vectors to date are usually derived from serotypes 2 (Ad2) and 5 (Ad5).

The biology of adenoviruses is relatively well understood because adenoviruses have played an essential part in molecular biology as experimental tools for elucidating various fundamental biological principles such as DNA replication, transcription, RNA splicing and cellular transformation. Adenoviral particles enter the cell during an infection through receptor-mediated endocytosis in which, according to the current view, interaction of the knob domain of the fiber protein with CAR mediates adhesion of the virus particle to the cell surface (Bergelson et al., Science 275, 1320-1323, 1997). In a second step there is internalization of the virus particle, for which interaction of the penton base with integrins plays an essential part (Wickham et al., Cell 73, 309-319, 1993). The internalization of the virion involves Arg-Gly-Asp (RGD) sequences in the penton base, which interact with the secondary host cell receptors, integrins αᵥβ3 and αᵥβ₅. After the particle has entered the cell, the viral genome obtains entry into the cell nucleus as a DNA-protein complex. The adenoviral infection cycle is divided into an early and a late phase, which are separated by the start of adenoviral replication (Shenk, in: Virology, Fields ed., Lippincott-Raven Publishing, Philadelphia, pp. 2111-2148, 1996). In the early phase there is expression of the early viral functions E1, E2, E3 and E4. The late phase is characterized by transcription of late genes, which are responsible for the expression of viral structural proteins and for the production of new viral particles.

All adenovirus vectors currently used in gene therapy are believed to have a deletion in the E1 region, where novel genetic information can be introduced. The E1 deletion renders the recombinant virus replication defective (Stratford-Perricaudet and Perricaudet, 1991). It has been shown that recombinant adenoviruses are able to efficiently transfer recombinant genes to the rat liver and airway epithelium of rhesus monkeys (Bout et al., 1994b; Bout et al., 1994a). In addition, researchers have observed a very efficient in vivo adenovirus mediated gene transfer to a variety of tumor cells in vitro and to solid tumors in animal models (lung tumors, glioma) and human xenografts in immunodeficient mice (lung) in vivo (Vincent et al., 1996a; Vincent et al., 1996b) and others (see, e.g., Haddada et al., 1993). The adenovirus vectors may be first-generation adenovirus vectors, second-generation adenovirus vectors, adenovirus vectors of large DNA capacity and/or deleted adenovirus vectors.

As mentioned, CAR is the adenovirus attachment receptor, which facilitates receptor mediated uptake into cells. Although CAR and D1 are well studied and have been used in vector targeting schemes, they have not been used to surface immobilize adenovirus vector for site-specific gene delivery. The main emphasis of CAR and D1 receptor research thus far has focused on the role of these proteins, and comparable receptor proteins in ligand binding and other biological mechanisms. CAR, for example, is involved in forming tight junctions between cells, and cell-to-cell signaling. The affinity of adenovirus for CAR appears to be a unique pathogenic property of this infective micro-organism.

In one embodiment of the present invention, CAR D1 is utilized to immobilize an adenoviral vector to a surface. Nonlimiting examples of such surfaces include metal surfaces, polymeric surfaces, argonometallic surfaces, or any surface to which the modified protein of the invention can be covalently attached. In another embodiment, the surface can be compositional materials made of various substances. In a preferred embodiment, the surface is a metal surface. In one embodiment of the invention, CAR D1 is attached to a surface by a linker of a surface modifier. A surface modifier suitable for the present invention is any compound that (i) can chemically coordinate with a surface, preferably a metal surface, and (ii) has a derivatizable functionality (e.g., a functional group) capable of reacting with a modified protein of the invention. The surface modifier of the invention also comprises a linker which is the part of the surface modifier remaining after reacting with the surface and the modified protein. Examples of such modifications can be found in Unites States Patent Application publication number 20030044408, filed on Jun. 14, 2002 entitled Surface Modification for Improving Biocompatibility, which is herein incorporated by reference. Specific examples of such surface modifiers include but are not limited to polybisphosphonates, aminobisphosphonates and polyamines. Aminobisphosphonates include polyaminobisphosphonates. Other surface coordinating compounds with side functionalities for branching attachment and amplification include any polymeric, oligomeric, or monomeric compound that contains groups capable of coordination to metal ions, such as phosphonic groups, hydroxamic groups, carboxylic groups, sulfonic residues, sulfinic groups and amino groups. The side functionalities capable of further reactions (when the modifier is already absorbed on the metal surface) could include amino or thiol groups (also in latent modifications, e.g., alkyldithio groups, which can be reduced to thiol groups immediately before the use), alkylating groups (maleimido, vinylsulfonyl, epoxy or iodoacetamido groups), and other groups suitable for the covalent attachment of proteins and at the same time, comparatively inert towards the coordination with the metal ions on the surface.

The polymeric backbone of the polymeric surface modifiers should be sufficiently stable in the aqueous surrounding, and can be represented by a chain consisting purely of carbon atoms (as for the polymers based on polyallylamine), or could incorporate heteroatoms (oxygen nitrogen, etc.) into the polymeric chain (e.g., polylysine, also with a part of lysine residues modified to insert chelating groups for better coordination to the metal). The polymeric surface modifier can be derived from a polyamine or other polymers. For example, it could be a polymer with pendant phosphonate or geminal bisphosphonate groups (for coordination with the metal ions on the surface) and alkyldithio groups as latent thiol functions for the subsequent protein tethering.

A chelating group is a chemical entity consisting of several units capable of coordination to the metal ions and positioned in close proximity to each other, so they could simultaneously bind the same metal ion, thus increasing the strength of the interaction. Chelating groups could contain units capable of formation of only metal-oxygen coordination bonds with the metal ions (geminal bisphosphonate, geminal or vicinal dicarboxylate, or hydroxamate), or they also could involve other atoms (e.g., iminodiacetate group, which in addition to the metal-oxygen bonds can also form metal-nitrogen bonds involving the tertiary amino group).

Coordination to the metal surface usually depends on pH and is suppressed in both strongly acidic and strongly alkaline media. Stronger chelators (like geminal bisphosphonate groups) could be used in wide regions of pH (approximately, from 2 to 12), whereas amino groups are much weaker towards the coordination with the metal surface, and probably, would be effective only in a narrow region of pH close to the value of pKₐ characteristic to them (ca. 10 for the aliphatic amino groups). All of these, alone or in combinations, would be suitable for coordination chemistry-based surface modifications. Preferably, the surface modifier is an aminobisphosphonate or a polyamine, such as polylysine or polyallylamine.

The number of CAR D1 binding sites can be amplified significantly by repeatedly adding the surface modifier and a crosslinker. A metal surface modified in this manner thus provides a greater potential for immobilization of biologically active molecules. For example, the number of CAR D1 binding sites can be enhanced to ultimately provide greater potential for gene transfer vector attachment. In a preferred embodiment, polyamines are reacted with the initial bisphosphonate coordination layer, a crosslinking agent then is added to the polyamines, and the polyamine and crosslinking steps are repeated to attain the desired level of protein-vector tethering.

As used herein, the term “layer” means a contiguous and non-contiguous deposit formed by a covalent bonding of a surface modifier, a modified protein or a gene transfer vector of the invention, or a composition comprising all three entities (a surface modifier, a modified protein and a gene transfer vector) to a surface. The term “coating”, as used herein, includes coatings that completely cover a surface, or portion thereof (e.g., continuous coatings, including those that form films on the surface), as well as coatings that may only partially cover a surface, such as those coatings that after drying leave gaps in coverage on a surface (e.g., discontinuous coatings). In some embodiments, the coating preferably forms at least one layer of deposit on the surface which has been coated, and is substantially uniform. However, when the coatings described herein are described as being applied to a surface, it is understood that the coatings need not be applied to, or that they cover the entire surface. For instance, the coatings will be considered as being applied to a surface even if they are only applied to modify a portion of the surface.

For example, the metal surface can be treated with either polyallylaminobisphosphonate (PAABP) or poly-bisphosphonates containing latent thiol groups to form a chemosorption layer with binding through coordination of the bisphosphonate groups. The primary amino groups of the PAA-BP chemosorption layer can be transformed with SPDP into the thiol-reactive pyridyldithio groups, which then can be used for the immobilization of thiol-containing proteins.

The chemosorption layers of poly-bisphosphonates with latent thiol groups can be reduced with tris(2-carboxyethyl)phosphine (TCEP) in aqueous buffered solutions, at pH ca. 5, for several minutes at room temperature. The immobilized thiol groups thus formed can then be reacted with thiol-reactive groups such as pyridyldithio or maleimido which have been pre-introduced into proteins by standard methods known in the art.

It is also possible to amplify the number of reactive functionalities attached to the chemosorption layer by using several variants of expansion chemistry. One such variant is the reaction of thiol groups on the chemosorption layer with a polymer containing multiple thiol-reactive groups, such as pyridyldithio groups as described above.

For example, pyridyldithio groups rapidly react with thiols in both aqueous (pH 5 to 8) and non-aqueous media, forming stable disulfide linkages. By using a large excess of the PAA-pyridyldithio polymers, most of pyridyldithio groups of the amplification polymer will remain unreacted, and can be later used for the immobilization of thiol-containing proteins. The polymers with multiple pyridyldithio groups are prepared from reactions of SPDP with polymeric amines such as polyallylamine and polyethyleneimine. These polyamines, in their “free base” form, can easily dissolve in non-aqueous solvents (dichloromethane or a mixture of dichloromethane and isopropanol) and smoothly react with SPDP at 0-20° C. The reactions are typically complete in less than 30 min, and no side-reactions (hydrolysis of succinimidyl ester, or degradation of pyridyldithio group) occur. Modified polymers prepared in this manner can be purified from non-polymeric impurities (N-hydroxysuccinimide, and sometimes, an excess of SPDP) by extraction with suitable solvents (methanol or isopropanol).

Using sub-stoichiometrical amounts of SPDP followed by the neutralization of unreacted amino groups with a suitable acid (e. g., HCl), it is also possible to react only a fraction of the amino groups with SPDP, thus obtaining positively charged water-soluble polymers with pyridyldithio groups.

Another variant in multiplying the number of reactive groups on the metal surface involves the reaction of PAABP on the metal surface with a suitable homobifunctional (or polyfunctional) amino-reactive cross-linker in a non-aqueous medium followed by treatment with polyallylamine. To eliminate the possibility of hydrolysis, the cross-linker and the amplifier-polyamine should preferably be used in non-aqueous media (e.g., DMF). An organic base (like triethylamine) can be added as the activator of amino groups in the first step, whereas the reaction between immobilized succinimidyl ester groups with the polyamine-base does not require any such activation. Under these conditions, the aminolysis of succinimidyl ester groups is usually complete in a few minutes at room temperature.

In another embodiment of the present invention, the gene transfer vector comprises a therapeutic nucleic acid. A nucleic acid of the present invention can be any polynucleotide that one desires to transport to the interior of a cell. In this context, a “therapeutic polynucleotide” is a polymer of nucleotides that, when provided to or expressed in a cell, alleviates, inhibits, or prevents a disease or adverse condition, such as inflammation, and/or promotes tissue healing and repair (e.g., wound healing). The nucleic acid can be composed of deoxyribonucleosides or ribonucleosides, and can have phosphodiester linkages or modified linkages, such as those described below. The phrase “nucleic acid” also encompasses polynucleotides composed of bases other than the five that are typical of biological systems: adenine, guanine, thymine, cytosine, and uracil.

A suitable nucleic acid can be DNA or RNA, linear or circular and can be single- or double-stranded. The “DNA” category in this regard includes: cDNA; genomic DNA; triple helical, supercoiled, Z-DNA, and other unusual forms of DNA; polynucleotide analogs; an expression construct that comprises a DNA segment coding for a protein, including a therapeutic protein; so-called “antisense” constructs that, upon transcription, yield a ribozyme or an antisense RNA; viral genome fragments, such as viral DNA; plasmids and cosmids; and a gene or gene fragment.

The nucleic acid also can be RNA, for example, antisense RNA, catalytic RNA, catalytic RNA/protein complex (e.g., a “ribozyme”), an expression construct comprised of RNA that can be translated directly, generating a protein, or that can be reverse transcribed and either transcribed or transcribed and then translated, generating an RNA or protein product, respectively; transcribable constructs comprising RNA that embodies the promoter/regulatory sequence(s) necessary for the generation of DNA by reverse transcription; viral RNA; and RNA that codes for a therapeutic protein, inter alia. A suitable nucleic acid can be selected on the basis of a known, anticipated, or expected biological activity that the nucleic acid will exhibit upon delivery to the interior of a target cell or its nucleus.

The length of the nucleic acid is not critical to the invention. Any number of base pairs up to the full-length gene may be transfected. For example, the nucleic acid can be a linear or circular double-stranded DNA molecule having a length from about 100 to 10,000 base pairs in length, although both longer and shorter nucleic acids can be used.

The nucleic acid can be a therapeutic agent, such as an antisense DNA molecule that inhibits mRNA translation. Alternatively, the nucleic acid can encode a therapeutic agent, such as a transcription or translation product which, when expressed by a target cell to which the nucleic acid-containing composition is delivered, has a therapeutic effect on the cell or on a host organism that includes the cell. Examples of therapeutic transcription products include proteins (e.g., antibodies, enzymes, receptor-binding ligands, wound-healing proteins, anti-restenotic proteins, anti-oncogenic proteins, and transcriptional or translational regulatory proteins), antisense RNA molecules, ribozymes, viral genome fragments, and the like. The nucleic acid likewise can encode a product that functions as a marker for cells that have been transformed, using the composition. Illustrative markers include proteins that have identifiable spectroscopic properties, such as GFP and proteins that are expressed on cell surfaces (e.g., can be detected by contacting the target cell with an agent that specifically binds the protein).

A nucleic-acid category that is important to the present invention encompasses polynucleotides that encode proteins that affect wound-healing. For example, the genes egf, tgf, kgf, hb-egf, pdgf, igf, fgf-1, fgf-2, vegf, other growth factors and their receptors, play a considerable role in wound repair.

Another category of polynucleotides, coding for factors that modulate or counteract inflammatory processes, also is significant for the present invention. Also relevant are genes that encode an anti-inflammatory agent such as MSH, a cytokine such as IL-10, or a receptor antagonist that diminishes the inflammatory response.

Suitable polynucleotides can code for an expression product that induces cell death or, alternatively, promotes cell survival, depending on the nucleic acid. These polynucleotides are useful not only for treating tumorigenic and other abnormal cells but also for inducing apoptosis in normal cells. Accordingly, another notable nucleic-acid category for the present invention relates to polynucleotides that, upon expression, encode an anti-oncogenic protein or, upon transcription, yield an anti-oncogenic antisense oligonucleotide. In this context, the phrases “anti-oncogenic protein” and “anti-oncogenic antisense oligonucleotide” respectively denote a protein or an antisense oligonucleotide that, when provided to any region where cell death is desired, or the site of a cancerous or pre-cancerous lesion in a subject, prevents, inhibits, or reverses abnormal and normal cellular growth at the site or induces apoptosis of cells. Delivery of such a polynucleotide to cells, pursuant to the present invention, can inhibit cellular growth, differentiation, or migration in order to prevent movement or unwanted expansion of tissue at or near the site of transfer. Illustrative of this anti-oncogenic category are polynucleotides that code for one of the known anti-oncogenic proteins. Such a polynucleotide would include, for example, a nucleotide sequence taken or derived from one or more of the following genes: abl, akt2, apc, bcl2-alpha, bcl2-beta, bcl3, bcl-x, bad, bcr, brca1, brca2, cbl, ccndl, cdk4, crk-II csflr/fms, dbl, dcc, dpc4/smad4, e-cad, e2fl/rbap, egfr/erbb-1, elk1, elk3, eph, erg, ets1, ets2, fer, fgr/src2, flil/ergb2, fos, fps/fes, fra1, fra2, fyn, hck; hek, her2/erbb-2/neu, her3/erbb-3, her4/erbb-4, hras1, hst2, hstfl, ink4a, ink4b, int2/fgf3, jun, junb, jund, kip2, kit, kras2a, kras2b, ck, lyn, mas, max, mcc, met, mlh1, mos, msh2, msh3, msh6, myb, myba, mybb, myc, mycl1, mycn, nf1, nf2, nras, p53, pdgfb, pim1, pms1, pms2, ptc, pten, raft, rb1, rel, ret, ros1, ski, src1, tal1, tgfbr2, thra1, thrb, tiam1, trk, vav, vhl, waf1, wnt1, wnt2, wt1, and yes1. By the same token, oligonucleotides that inhibit expression of one of these genes can be used as anti-oncogenic antisense oligonucleotides.

Nucleic acids having modified internucleoside linkages also can be used in a composition according to the present invention. For example, nucleic acids can be employed that contain modified internucleoside linkages which exhibit increased nuclease stability. Such polynucleotides include, for example, those that contain one or more phosphonate, phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂—), dimethylene-sulfoxide (—CH₂—SO—CH₂—), dimethylene-sulfone (—CH₂—SO₂—CH₂—), 2′-O-alkyl, and 2′-deoxy-2′-fluoro-phosphorothioate internucleoside linkages.

For present purposes, a nucleic acid can be prepared or isolated by any conventional means typically used to prepare or isolate nucleic acids. For example, DNA and RNA can be chemically synthesized using commercially available reagents and synthesizers by known methods. For example, see Gait, 1985, in: OLIGONUCLEOTIDE SYNTHESIS: A PRACTICAL APPROACH (IRL Press, Oxford, England). RNA molecules also can be produced in high yield via in vitro transcription techniques, using plasmids such as SP65, available from Promega Corporation (Madison, Wis.). The nucleic acid can be purified by any suitable means, and many such means are known. For example, the nucleic acid can be purified by reverse-phase or ion exchange HPLC, size exclusion chromatography, or gel electrophoresis. Of course, the skilled artisan will recognize that the method of purification will depend in part on the size of the DNA to be purified. The nucleic acid also can be prepared via any of the innumerable recombinant techniques that are known or that are developed hereafter.

A suitable nucleic acid can be engineered into a variety of known host vector systems that provide for replication of the nucleic acid on a scale suitable for the preparation of an inventive composition. Vector systems can be viral or non-viral. Particular examples of viral vector systems include adenovirus, retrovirus, adeno-associated virus and herpes simplex virus. As stated, in a preferred embodiment of the present invention, an adenovirus vector is used. A non-viral vector system includes a plasmid, a circular, double-stranded DNA molecule. Viral and nonviral vector systems can be designed, using known methods, to contain the elements necessary for directing transcription, translation, or both, of the nucleic acid in a cell to which it is delivered. Methods which are known to the skilled artisan can be used to construct expression constructs having the protein coding sequence operably linked with appropriate transcriptional/translational control signals. These methods include in vitro recombinant DNA techniques and synthetic techniques. For in stance, see Sambrook et al., 1989, MOLECULAR CLONING: A LABORATORY MANUAL (Cold Spring Harbor Laboratory, New York), and Ausubel et al., 1997, CURRENT PROTOCOLS IN MOLECULAR BIOLOGY (John Wiley & Sons, New York).

A nucleic acid encoding one or more proteins of interest can be operatively associated with a variety of different promoter/regulator sequences. The promoter/regulator sequences can include a constitutive or inducible promoter, and can be used under the appropriate conditions to direct high level or regulated expression of the gene of interest. Particular examples of promoter/regulatory regions that can be used include the cytomegalovirus (CMV) promoter/regulatory region and the promoter/regulatory regions associated with the SV40 early genes or the SV40 late genes. Preferably, the human CMV promoter is used, but substantially any promoter/regulatory region which directs high level or regulated expression of the gene of interest can be used.

It also is within the scope of the present invention that the employed nucleic acid contains a plurality of protein-coding regions, combined on a single genetic construct under control of one or more promoters. In one embodiment, the modified protein is a fusion protein comprising a fragment of CAR and a receptor targeting ligand. The two or more protein-coding regions can be under the transcriptional control of a single promoter, and the transcript of the nucleic acid can comprise one or more internal ribosome entry sites interposed between the protein-coding regions. Thus, a myriad of different genes and genetic constructs can be utilized.

The present invention describes a composition comprising a modified protein and a surface, preferably a metal surface, to which functionalized bisphosphonates are covalently linked. In a preferred embodiment, the surface modifier is an aminobisphosphonate. The coordination of functionalized bisphosphonates provides a high affinity method of directly loading proteins with suitable reactive groups, like thiol, carboxy, or amino functions, such as proteins or polypeptides via cross linkers, to a metal support. By virtue of the chelating bisphosphonate groups, the amino bisphosphonate molecules coordinate nearly irreversibly to various metal ions, such as those of iron, chromium and nickel. In another preferred embodiment, the reactive group is a thiol.

A preferred embodiment of the method contemplates the use of a polyamine surface modifier. Polyamines can chemically coordinate with a metal surface and tightly bind steel, for example, in the same manner as aminobisphosphonates. The amino groups of the polyamines that do not coordinate with the metal surface can be derivatized.

Another preferred embodiment of the method employs a polybisphosphonate that comprises multiple bisphosphonate residues and multiple reactive functional groups. Increasing the number of bisphosphonate groups enhances binding affinity to the metal surface. Consequently, a greater number of functionalities can be used for the immobilization of proteins, and thus provide greater potential for nucleic acid attachment. In a preferred embodiment, the aminobisphosphonate is a polybisphosphonate.

For example, pamidronic acid (3-amino-hydroxypropylidene-1,1-bispho-sphonic acid), an amino-bisphosphonic acid, was converted into its potassium salt, in distilled water, to increase the solubility. This pamidronate solution was reacted with the metallic surface, thereby forming coordination bonds between the bisphosphonate groups and the metal cationic sites. In this way, the amino groups were introduced onto the metallic surface, where they could be used as functional groups for further chemical binding. Preferably, the metal surface is a stainless steel surface. A heterobifunctional crosslinker such as SPDP (N-succinimidyl-3-(2-pyridyldithio)-propionate) can be employed to link the chemosorbed layer to a protein or polypeptide. However, any crosslinker able to react both with the active functional groups of the chemosorption layer and those of an antibody can be used. Examples of suitable crosslinkers include SPDP, HBVS (1,6-hexane-bis-vinylsulfone), EMCS (N-[.epsilon.-maleimidoca-proyloxy]succinimide ester), BMH (bis-maleimidohexane), DPDPB (1,4-di-[3(2-pyridyldithio)-propionamido]butane, and other thiol-to-thiol crosslinkers can be used to provide the chemosorption layer with thiol groups.

The SPDP crosslinker reacts with the amine group of bisphosphonate-modified polyallylamine (PAA-BP), chemically linking the pyridyldithio groups of SPDP to the metallic surface. This residue then reacts with a modified protein, which covalently links the molecule to a metal support. In one preferred embodiment of the invention, the modified protein is CAR D1. Still more preferred, the CAR D1 is thiol modified. Such a thiol modified molecule is a substance that comprises a thiol group or has been modified to comprise a thiol group, such as thiol modified protein and peptide.

A bisphosphonate-modified polyallylamine (PAA-BP) can be prepared by the nucleophilic addition of the polyallylamine amino groups to the activated double bond of vinylidene-bisphosphonic acid (VBP).

An additional variant of linking proteins to the metal surface contemplates the use of monomeric or polymeric bisphosphonates already containing reactive groups. Examples of these reactive groups are vinylsulfonyl and maleimido groups, which are inert toward bisphosphonate groups, but reactive toward suitable groups on a protein such as thiol groups.

The present invention also contemplates preparing a composition by (i) modifying a surface and (ii) linking a modified protein or polypeptide to said surface. The surface is modified as discussed above. Preferably, the surface is a metal surface. In a preferred embodiment, the aminobisphosphonates are used as the surface modifier. In a more preferred embodiment, polyamines are used. In a most preferred embodiment, both aminobisphosphonates and polyamines can be used together to modify the metal surface. In one embodiment, the modified protein is CAR D1. In a preferred embodiment, the CAR D1 is thiol modified. In another preferred embodiment, a gene transfer vector is bound to the CAR D1.

The invention also contemplates a method for delivering the composition to a cell, comprising (A) exposing a cell to a complex comprised of (i) a modified protein bound to a gene transfer vector, (ii) a metal surface and (iii) a linker to which said modified protein and metal surface are chemically coordinated. In a preferred embodiment the gene transfer vector is an adenovirus vector. The composition can then be used to deliver a nucleic acid to the interior of a cell in need of gene therapy.

If viral vectors are tethered to the metal surface, addition of a cationic macromolecule is not necessary for efficient nucleic acid delivery. Viral vectors have been regarded as the most efficient system, and recombinant replication-defective viral vectors have been used to transduce cells both in vitro, in vivo and ex vivo. Such vectors have included retroviral, adenovirus, adeno-associated viral vectors and herpes viral vectors. Cells can be infected with viral vectors by known methods.

In a preferred embodiment, the metal surface is associated with a metal support. In this description, “metal support” denotes a uniform, solid homogenous or heterogenous material support, or a network of supporting structures suitable for gene therapy in accordance with the present invention. The metal support can be any structure having a metal surface, including preferably medical devices. A “medical device” is any tool, mechanism, or apparatus that can be used during medical intervention, including but not limited to surgical implants, surgical sutures, and prostheses. The medical device may be internal or implantable, or it may be external such that it is placed on the skin.

Illustrative of suitable metallic materials are stainless steel, MP35 stainless steel, aluminum oxide, platinum, platinum alloys, elgiloy, tivanium, vitallium, titanium, titanium alloys, Nitinol (nickel-titanium alloy), chromium alloys and cobalt based alloys and the like. Oxides of these metals and alloys can also be used. In a preferred embodiment, a medical device with a stainless steel surface, such as a stent, is also preferred. The metallic surface can be modified to facilitate attachment of the biologically active molecule without the use of polymers and other coatings. In another embodiment, the surface comprises only a percentage of metal.

Medical devices appropriate for the gene delivery system in the present invention include, but are not limited to, heart valves, wire sutures, temporary joint replacements and urinary dilators. Other suitable medical devices for this invention include orthopedic implants such as joint prostheses, screws, nails, nuts, bolts, plates, rods, pins, wires, inserters, osteoports, halo systems and other orthopedic devices used for stabilization or fixation of spinal and long bone fractures or disarticulations. Other devices may include non-orthopedic devices, temporary placements and permanent implants, such as tracheostomy devices, intraurethral and other genitourinary implants, stylets, dilators, stents, vascular clips and filters, pacemakers, wire guides and access ports of subcutaneously implanted vascular catheters.

Viral gene vectors, as used in prior art methods, have the drawback that they often cannot be delivered to a selected tissue in a specific, localized manner. Instead, many prior art methods of administering viral vectors result in vector being dispersed systemically or to tissues that adjoin, or are in fluid communication with, the desired target tissue. The inability of such methods to localize viral vector reduces the utility of the methods, because non-localized viral vector can transfect unintended tissues, elicit immune responses, be rapidly excreted from the body, or otherwise suffer diminished transfection ability. A critical need remains for gene therapy methods that can efficiently deliver viral vectors to targeted cell populations. Others working in the field have concentrated on attempting to specifically target adenovirus vectors to a particular cell type, for example by attaching a specialized receptor ligand to the vectors (Tzimagiorgis et al., 1996, Nucl. Acids 24:3476-3477). In one embodiment of the present invention, an adenoviral vector is immobilized to a medical device, whereby the adenoviral vector infects a specific population of cells surrounding the device. The binding of CAR D1 to the adenoviral vector facilitates cell entry as well through binding of integrins on the surface of specific cells.

The adenoviral fiber protein is also involved in determining which types of cells will be efficiently infected when contacted with an adenovirus. Accordingly, recombinant fiber proteins have been made which have affinity for cellular receptors other than CAR (see Bergelson et al., Science, 275, 1320-1323 (1997), and Hong et al., EMBOJ., 16, 2294-2306 (1997)). In another embodiment, adenoviral fiber protein receptors or fragments thereof may be used to immobilize an adenoviral vector to a medical device.

In another embodiment, the modified protein is a fusion protein. In a preferred embodiment, the fusion protein comprises a fragment of CAR ligated to a receptor targeting ligand, thereby forming a fusion protein that is able to target a cell through binding to a cellular receptor (See O. Nyanguile et al., Gene Ther. August 2003; 10(16):1362-9 which is herein incorporated by reference). In one embodiment, the receptor targeting ligand is a protein or a polypeptide. In a preferred embodiment, the fragment of CAR is an extracellular domain of CAR or an immunoglobulin D1 domain of CAR. In one embodiment, the receptor targeting ligand is derived from apolipoprotein E, transferrin, one of the vascular endothelial growth factors (VEGFs), one of the transforming growth factor(TGF)-betas, on of the fibroblast growth factors (FGFs), and RGD containing peptide, or folic acid or virtually any ligand-receptor pair entity. The receptor targeting ligand is responsible for targeting the vector to a specific cell by binding to said cell's receptor. In another embodiment, the receptor is a lipoprotein receptor or receptors for the following: transferrin, one of the VEGFs, one of the TGF-betas, one of the FGFs, a recombinant integrin receptor protein, a folic acid receptor, a folate receptor, or virtually any ligand receptor pair entity.

In one embodiment, the fusion protein is generated by intein-mediated protein ligation. In a further preferred embodiment, the modified protein further comprises an intein splicing element. Inteins are catalytic domains involved in protein splicing. Protein splicing involves the self-catalyzed excision of an intervening sequence, the intein, from a precursor protein, with the concomitant ligation of the flanking extein sequences to yield a new polypeptide.¹ The discovery of inteins as protein-splicing domains has led to the development of a ligation technique, intein-mediated protein ligation, that allows for the versatile attachment of molecules to native proteins.² Intein-mediated protein ligation takes advantage of the catalytic activity of the intein to generate an activated thioester bond at the C-terminus of the protein of interest, to which virtually any molecular probes can be ligated. Examples include the incorporation of noncoded amino acids into a protein sequence,³ synthesis of cytotoxic proteins,⁴ segmental labeling of proteins for NMR analysis,⁵ the addition of fluorescent probes to create biosensors⁶ and the synthesis of glycoproteins.⁷ In one embodiment, this invention shows that intein-mediated protein ligation can be utilized to attach an array of targeting ligands that differ in size and nature to an Ad binding moiety, that is, peptides, proteins and small molecules.

Viral vectors are able, to a limited degree, to deliver proteins and other therapeutic molecules to the cells that the virus vectors transfect. Such proteins and other therapeutic molecules can be incorporated passively and non-specifically into viral vector particles. Alternatively, certain viral vectors specifically incorporate fusion proteins comprising a protein having a polypeptide viral packaging signal fused therewith.

Previous work has shown that anti-adenovirus antibodies can be used to tether gene vectors on the surface of implantable medical devices, such as vascular stents. The present invention represents a major advance on this approach in which a fragment of the human adenovirus receptor is used as a binding entity to immobilize adenovirus on the surface of an implantable medical device. As stated, the present invention utilizes CAR D1 to immobilize an adenoviral vector to the surface of a medical device. In a preferred embodiment, the CAR D1 is recombinant, and can, therefore, be produced in bacterial cultures with simple low cost purification procedures known to those of ordinary skill in the art. Thus, bacterial bioreactors can be established for large-scale production of D1 or comparable constructs, with appropriate customized purification systems for rapidly obtaining bulk quantities. In addition, immobilization of CAR D1 on a solid surface increases dramatically the affinity of D1 toward the knob domain of adenovirus (expected Kd=1 nM), when compared to D1 in solution (expected Kd=20 nM). Thus, the present invention should in principle provide a true biomimetic of the CAR protein.

The invention will be illustrated in more detail with reference to the following Examples, but it should be understood that the present invention is not deemed to be limited thereto.

### EXAMPLES

### Example 1

D1 Immobilization and Cell Culture

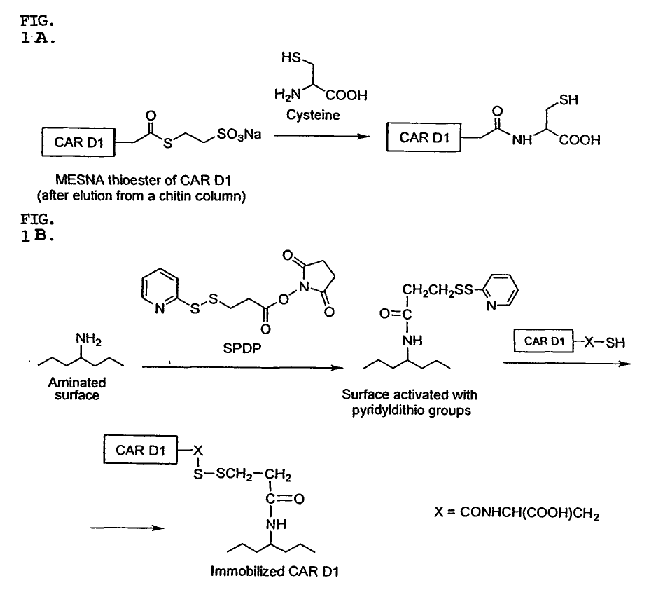

The D1 plasmid pTWIN-D1 and the CAR D1 activated thioester were prepared as already described (O. Nyanguile et al., Gene Ther. August 2003; 10(16):1362-9). The treatment with cysteine transformed the thioester into a thiol-containing form of D1 (D1-Cys) suitable for the further immobilization (FIG. 1A). Fifty mM boric buffer (pH 9) was supplemented with 10 mM EDTA and degassed. Three hundred μl of this buffer was added to 800 μl of D1 solution (final pH 8.5). L-cysteine (1.4 mg or 10 mM final concentration) was added and the reaction mixture was kept under argon atmosphere at 4° C. under mild shaking for 68 hours. D1-Cys was then purified using a desalting column equilibrated with degassed PBS/10 mM EDTA. The fraction with highest protein content was collected and kept on ice under argon atmosphere prior to further use. D1-Cys was covalently attached by disulfide bond formation on a surface activated with highly thiol-reactive 2-pyridyldithio (PDT) groups (FIG. 1B).

Four stainless steel meshes were pretreated with 0.5 N nitric acid and isopropanol (15 min each), washed in deionized distilled water (DDW) and incubated in 3% solution of polyallylamine bisphosphonate (pH 5.5) for at 60° C. for 4 hours. Then, the meshes were washed in DDW and reacted with SPDP (20 mg/ml; DMF:PBS=3:1) for 90 min at RT under moderate shaking.

The meshes were then washed in degassed PBS/EDTA and placed into CAR-D1-Cys conjugate solution (1 ml). Fifty mg BSA was added to the reaction mixture. The conjugation was allowed to run for 16 hours at room temperature (RT) under mild shaking. The meshes were washed in PBS and incubated with 1.6×10¹¹ particles of Cy3-labeled AdV-GFP in 500 μl in 5% BSA/PBS for 20 hours at RT under mild shaking. Additional two meshes coupled with antiknob Ab were prepared for the efficacy control purposes. The antiknob Ab primed meshes were then exposed to the same suspension of Cy3-labeled AdV-GFP under identical conditions.

Finally, the meshes were washed in PBS and examined under a fluorescent microscope. All four meshes of the meshes that were treated according to the CAR-D1 conjugation procedure exhibited uniform intensive fluorescence, which was only slightly inferior to the fluorescence emitted from the meshes with antiknob Ab-mediated virus tethering (data not shown). After confirming AdV tethering to the surface, the meshes were placed into confluent cultures of HEK 293 and A10 cells. Transduction was assessed 20 hours after mesh placement. In 293 cells CAR-D1 -conjugated meshes resulted in very high transduction of the cells that were in immediate proximity to the mesh. The transduction levels were much higher than those achieved with the antiknob Ab conjugation based strategy (data not shown).

### Example 2

Adenovirus Immobilization on Various Materials

This experiment was designed as a quantitative comparison of three different materials (stainless steel, Co/Cr and nitinol) in terms of the amount of Ad immobilized on Ab- and D1-tethered metal surface.

Four stainless steel foils, four Co/Cr coupons and four nitinol samples were cleaned with isopropanol and 1 N nitric acid, washed and incubated in 3% PAABP at 75° C. for 4 hours. The samples were then washed with DDW and reacted with SPDP (15 mg/ml in DMF:PBS=5:1) for 1 hour at RT under intensive shaking. The specimens were then washed and reacted with 450 μl of desalted D1, which was filtered through the polyacrylamide gel-filled column; the fraction 2.5-5.5 ml was collected in degassed PBS/EDTA and diluted with 3 ml of degassed PBS/EDTA. The final concentration of D1 was 0.3 mg/ml. The conjugation was carried out overnight at 37° C. under argon atmosphere.

Next, 500 μl of Cy3Ad-GFP was diluted to 18.5 ml with PBS. 1.5 ml aliquots were added to 12 individual samples, while 500 μl of the diluted Cy3Ad formulation was put aside as a reference. The reaction of immunoconjugation was allowed to run for 3 hours at RT under mild shaking.

The assessment of Cy3 Ad in the supernatants allows determining the amount of attached virus as the difference between the readings of non-depleted Ad and reading in respective supernatants. Absolute concentration of virus in non-depleted formulation was determined by the spectrophotometry at 260/280 nm. The extent of depletion was assessed by fluorimetry at 540/580 nm.

In an independent experiment, two stainless steel foils, two Co/Cr coupons and 2 nitinol specimens were pretreated with isopropanol/chloroform and 1N nitric acid and reacted with 3% PAABP under intensive shaking for 4 hours at 75° C. Next, the samples were washed in DDW and reacted with SPDP (15 mg/ml; DMF:PBS=5:1) for 50 min at 32° C. under intensive shaking. After washing, SPDP-treated samples of each type were reacted with reduced anti-knob Ab. The latter was eluted into degassed PBS/EDTA (1 mg of Ab was reduced with 20 mg/ml of 2-mercaptoethylamine for 60 min at RT under shaking and passed through the desalting column. The fraction 2.5-5.5 ml was diluted twice with degassed PBS and added to the metal samples). The conjugation was allowed to run for 18 hours, after which the samples were washed with PBS for 3 hours and individually incubated in 1 ml of Cy3Ad-iNOS suspension. Namely, 55 μl of the stock (total amount of virus is 7×10¹⁰ particles) was suspended in 6.945 ml of PBS. One ml aliquots of the suspension were added to six samples and additional ml was put aside to serve a non-depleted control. Following a 3 hour incubation at 30° C. under intensive shaking, partially depleted, suspension samples were assessed for Cy3 fluorescence using non-depleted sample as 100% value.

The following data represent a vector binding capacity comparison of specific antibody versus D1-receptor. These results demonstrate that D1 mediates two to four fold greater adenovirus binding on various alloys. Data are Ad (×10⁹) bound per cm² of the surface.

| | The amount of Ad (×10⁹) bound per cm² | | |
|---|---|---|---|
| | of the surface | | |
| Linking Agent | Stainless steel | Nitinol | Co/Cr alloy |
| Anti-knob Ab | 4.3 ± 0.34 | 5.27 ± 0.1 | 3.52 ± 1.13 |
| D1 | 10.8 ± 0.6 | 11.6 ± 0.42 | 15.1 ± 1.37 |

### Example 3

Ad-GFP Expression in a Rat Carotid Artery

Four stainless steel stents were pretreated subsequently with isopropanol (15 min) and 1N nitric acid (15 min) and heated at 220° C. (2 hours). Then the specimens were reacted with 3% PAABP for 5 hours at 60° C. After washing (DDW, ×3) the stents were reacted with SPDP (22 mg/ml; DMF:PBS=3:1) for 85 min at RT. In parallel 2 mg of antiknob Ab was reduced by 2-mercaptoethylamine (final concentration is 13.8 mg/ml) for 75 min at 37° C.

Then, reduced antiknob Ab and D1-Cys were individually purified by gel-filtration via polyacrylamide-filled column equilibrated with degassed PBS/10 mM EDTA. For both, protein fractions comprising 2.5-5 ml were collected and mixed with 0.83 ml of 20% BSA/degassed PBS. Two stents were allocated for the each group (antiknob Ab and CAR-D1). The reactions were allowed to run for 16 hours under mild shaking at RT under argon atmosphere.

Next, the stents were washed with PBS and individually treated in 1 ml of 5% BSA/PBS+100 μl of AdV-GFP (IHGT prep) for 10 hours at RT under mild shaking. Stents were mounted on NINJA catheters, manually crimped and left in the same virus suspension at 4° C. for additional 14-18 hours and washed with PBS immediately before use.

Under ketamine/xylosine anesthesia, the stents were deployed in the middle segments of the common carotid arteries in 4 rats. The left common carotid artery and the proximal segments of external and internal carotid arteries were exposed using sharp and blunt dissection of skin, fascia and muscles. The external carotid artery was tied off permanently, while a sliding knot was placed on the origin of the internal carotid artery to temporarily exclude it from circulation. A bulldog clamp was placed in the mid-segment of the common carotid artery. At that point, the animals were injected with a 100 U of heparin via tail vein. In the central part of the exposed external carotid artery, a ca. 1 mm longitudinal incision was made. A 2 F Fogarty catheter was introduced into external carotid artery and rapidly advanced into the common carotid. The balloon was inflated with saline until there was a feeling of slight resistance to the movement, at which time the catheter was dragged back to the site of insertion. This procedure was repeated three times to denude endothelium. After completion of denudation, the Fogarty catheter was withdrawn. A Cordis (Cordis Corp., Miami Lakes, Fla.) angioplasty NINJA catheter (20 mm-long balloon, 1.5 mm inflated diameter) bearing a crimped 7 mm stent was inserted into common carotid artery. The stent was deployed at the mid-segment of the common carotid artery at 10 atm for 1 min. The catheter was then withdrawn, the knot around the internal carotid was loosened and the clamp from the common carotid artery was finally removed. The circulation in the carotid system was restored, though bypassing the external carotid artery. The skin wound was closed using surgical clips.

Rats were sacrificed three days after surgery. All four arteries were patent. The stents were removed and studied by fluorescent microscopy. Stent-associated tissue had a much higher number of transduced cells in the case of D1-tethering in comparison with antiknob Ab-tethering (data not shown).

Arteries were embedded in OCT and cut with the section thickness of 6 μm. They were examined by fluorescence microscopy after Evans Blue/DAPI counterstaining (data not shown). Green fluorescence (GFP) was detected in the media and the adventitia. Elastic fiber of the media appears red and cell nuclei appear blue.

GFP immunochemistry of arterial sections treated by DI-tethered stents showed very significant staining of media (data not shown). The OCT cryosections were thawed, washed in water, fixed in 4% buffered formalin for 3 min, permeabilized with 0.02% Triton 100. Next, the sections were microwave-treated in the boiling citric buffer (pH 6) for epitope retrieval (5 min). After cooling, the slides were incubated in 10% goat serum for 20 min to eliminate background staining.

The sections were then exposed to a polyclonal rabbit anti-GFP Ab (Abcam; ab290) at 1:200 dilution in 1% BSA/PBS for 18 hours at 4° C. After PBS washing, arterial sections were stained with secondary goat anti-rabbit Cy3-tagged Ab (Jackson ImmunoResearch, West Grove, Pa.; 1:250 dilution in 1% BSA/PBS) for 45 min. Finally, the slides were washed with PBS and mounted using DAPI-containing medium (data not shown).

### Example 4

Ad-GFP Expression in Pig Coronary Arteries

Six stainless steel stents and two indicator meshes were pretreated subsequently with isopropanol (15 min), 1 N nitric acid (15 min) and heating at 220° C. (1.5 hours). Then the specimens were reacted with 3% PAABP for 4 hours at 60° C. After washing (DDW ×3), the stents were reacted with SPDP (20 mg/ml; DMF:PBS=3:1) for 75 min at RT.

In parallel, 1 ml of cysteinated CAR D1 (2.2 mg) was desalted (2.7-5.4 ml) from the excess of cysteine in degassed PBS/10 mM EDTA (final concentration of CAR D1 was ca. 0.75 mg/ml). The purified D1 was added to the specimens (without BSA) and reacted with the SPDP-primed metal surfaces for 16 hours at RT under argon atmosphere.

The next day the specimens were washed and the stents were reacted with 150 μl of AdV-GFP suspended in 1.5 ml of 5% BSA. The meshes were reacted with 75 μl of Cy3AdV-GFP in 750 μl of 5% BSA. Immune conjugation was carried for 16 hours at RT under shaking.

Next, the specimens were washed with PBS, and the meshes were observed under a fluorescent microscope and pictures were taken. The specimens were moderately fluorescent. Four of six stents were uneventfully deployed in the LAD and Cx arteries of domestic pigs using femoral approach for coronary catheterization.

The pigs were sacrificed on day 7 after stent deployment. Four stented segments were retrieved, and the stents were removed. Stents were flattened and observed under the fluorescent microscope. All of them had clusters of brightly fluorescent cells attached to stent struts. Pictures were taken prior to and after DAPI staining.

Arteries were embedded in OCT and cut with the section thickness of 6 μm. The OCT cryosections were thawed, washed in water, fixed in 4% paraformaldehyde for 3 min, and permeabilized with 0.02% Triton 100. Next, the sections were microwave-treated in the boiling citric buffer (pH=6) for epitope retrieval (5 min). After cooling the slides were incubated in 10% goat serum for 20 min to eliminate background staining.

The sections were then exposed to polyclonal rabbit anti-GFP Ab (Abcam, Cambridge, Mass.; ab290) at 1:200 dilution in 1% BSA/PBS for 18 hours at4° C. After washing with PBS, the arterial sections were stained with secondary goat anti-rabbit biotin-labeled Ab (Jackson ImmunoResearch; 1:150 dilution in 1% BSA/PBS) for 1 hour followed by the exposure to the avidin/biotin/peroxidase complex (Vector Laboratories, Burlingame, Calif., ABC Elite kit). Finally the color was developed by the diaminobenzidine (Sigma Chemical Co., St. Louis, Mo., DAB kit). The slides were dehydrated by serial alcohol/xylene washings and permanently mounted.

GFP immunochemistry of pig coronary sections treated by D1-tethered stents demonstrated intensive intimal staining with scattered staining in the media and adventitia that was not present when the primary Ab was omitted (data not shown).

### Example 5

Targeting of a Gene Transfer Vector to Specific Cell Types

Materials and Methods

Production and Purification of D1

A DNA fragment encoding the D1 domain of CAR (D1: amino acid residues 15 to 140) was generated by PCR as a NdeI-SacI DNA fragment and cloned into the pTWIN-MBP1 vector (New England Biolabs, Beverly, Mass.). This cloning strategy generated a pTWIN-D1 expression construct encoding the D1-GyrA-CBD fusion protein, which contains a 24 amino-acid spacer between CAR D1 and the Mxe GyrA mini-intein. pTWIN-D1 was transformed into the Escherichia coli B121(DE3) strain and cells were grown at 37° C. in 1 liter of LB broth (100 μg/ml ampicillin) to an optical density of 0.4 at 600 nm, after which the culture was induced with 0.3 mM isopropyl-β-D thiogalactopyranoside (IPTG) and transferred to 18° C. Cells were harvested after overnight incubation, lysed by sonication in Buffer E (20 mM Tris-HCl (pH 7.5), 100 mM NaCl), and centrifuged at 30,000 g for 30 min. The supernatant was loaded on a column packed with chitin resin (5 ml bed volume) and equilibrated in Buffer E. Unbound proteins were washed from the column with 80 ml of Buffer E. Intein-mediated generation of the CAR D1 C-terminal thioester was initiated by quickly flushing the column with 15 ml of a 100 mM solution of 2-mercaptoethanesulfonic acid (MESNA, Aldrich, Milwaukee Wis.) in Buffer E, after which the flow was stopped and the cleavage reaction was allowed to proceed overnight at 4° C. CAR D1 was eluted with 7 ml of 100 mM MESNA in Buffer E and concentrated on a Centriprep 3 centrifugal filter device. The protein concentration was determined using the Coomassie plus protein assay (Pierce, Rockford Ill.) using BSA as a protein standard.

Production and Purification of Targeting Ligands

Synthesis of apolipoprotein E (apoE) Peptide

The apoE targeting peptide:

CLRKLRKRLLRDADDLLRKLRKRLLRDADDLGSDDDDD-NH2⁸ (SEQ ID NO:1) was synthesized by standard solid phase peptide synthesis on a Pioneer Peptide Synthesizer (Applied Biosystems, Foster City Calif.), using Fmoc/Boc(t-Bu)/HATU chemistry on a Novasyn TGR resin (Calbiochem-Novabiochem Corp., San Diego Calif.). Following deprotection from the solid support, the peptide was purified by reversed-phase HPLC and freeze-dried. HMRS (ESI) for C₁₉₀H₃₃₅N₆₇O₆₁S, 4566.4(MH+), calcd 4567.23.

Expression and Purification of FGF2

A cDNA fragment encoding basic fibroblast growth factor FGF2 (GenBank Accession No. NM 002006) was generated by PCR as a SapI-PstI fragment and cloned into the pTWIN2 vector (New England Biolabs, Beverly Mass.). Additional nucleotides encoding the sequence Asn-Cys-Arg required for expressed protein ligation,⁹ were included after the SapI site as recommended by the manufacturer (New England Biolabs, IMPACTTM-TWIN). The SapI site present in the FGF2 cDNA was eliminated prior to PCR by site directed mutagenesis using a mutation that maintained identical codon translation (QuickChange™ site directed mutagenesis kit, Stratagene, La Jolla, Calif.). The primer sequence was 5′-CGGGGTCCGGGAGAAAAGCGACCCTCACATC-3′ (SEQ ID NO:2). This cloning strategy generated a pTWIN2-FGF2 expression construct encoding the CBD-Ssp-FGF2 fusion protein. Protein expression was performed as described for D1-GyrA in 2 liters of LB broth. Cells were lysed in Buffer C [20 mM Tris-HCl (pH 8.5), 500 mM NaCl, 1 mM EDTA] and the cleared cell lysate was loaded onto 7.5 ml (bed volume) of a chitin resin column. After washing with 120 ml Buffer C, intein-mediated generation of the FGF2 protein was initiated with a rapid flush-through of 22.5 ml Buffer D (20 mM Tris-HCl (pH 7.0), 500 mM NaCl, 1 mM EDTA). After 48 h incubation at room temperature, FGF2 was eluted with 14 ml Buffer D and concentrated on a Centriprep 3.

Derivatization of Folic Acid

In preparation for ligation to CAR D1, folate was derivatized in three steps. First, a cysteine residue necessary for the ligation step to D1 was attached to an ethylenediamino linker to generate N-<-trityl-S-trityl-L-cysteine-ethylenediamine. This reagent was made as follows: Ethylenediamine (97.35 ml, 110 mmol) was added to a solution of Trt-Cys(Trt)-OSu (860 mg, 1.1 mmol, Novabiochem, San Diego, Calif.) in 23 ml of dichloromethane. After stirring for 5 h at room temperature, the reaction mixture was diluted with dichloromethane (35 mL), after which the organic phase was washed extensively with water and dried over Na₂SO₄. Evaporation yielded 649 mg of a yellowish amorphous powder. The molecular weight of the product was characterized by fast-atom bombardment high mass resolution spectroscopy: HRMS (FAB) for C₄₃H₄₁N₃O₅, 648.0 (MH+), calcd 648.9.

The second step in the synthesis joined free folate to the ethylenediamino linker to generate N-α-trityl-S-trityl-L-cysteine-ethylenediamine-α,γ-folate. This reaction was carried out as follows: N-α-trityl-S-trityl-L-cysteine-ethylenediamine (608 mg, 0.94 mmol), O-(7-Azabenzotriazol-1-yl)-N,N,N′,N′-tetramethyluronium hexafluorophosphate (HATU, 356.8 mg, 0.94 mmol, Applied Biosystems, Foster City, Calif.) and folic acid (448 mg, 0.94 mmol) were combined and dissolved in 27 mL dry DMSO. Following addition of N,N-diisopropylethylamine (660 μl, 3.76 mmol), the reaction mixture was stirred overnight, after which the product was precipitated with an excess of acetone/ether (30:70). The precipitate was centrifuged 10 min at 3,000×g, washed twice with acetone/ether (30:70) and dried under vacuum to yield 655 mg of a yellowish amorphous powder. HRMS (FAB) for C₆₂H₅₈N₁₀O₆S, 1071 (M), calcd 1071.28.

The last step in the synthesis involved the removal of the cysteine protecting groups to generate L-cysteine-ethylenediamine-α,γ-folate. This was carried out as follows: N-α-trityl-Strityl-L-cysteine-ethylenediamine-αγ-folate (203 mg, 0.19 mmol) was dissolved in 10 mL TFA and triisopropylsilane (155 μL, 0.758 mmol) was added. The reaction mixture was stirred for 1 hour at room temperature and the product was precipitated with an excess of acetone/ether (30:70). The precipitate was centrifuged for 10 min at 3,000 g, washed twice with acetone/ether (30:70) and dried under vacuum to yield 130 mg of an amorphous orange powder. The final product was analyzed by fast-atom bombardment high mass resolution spectroscopy: HRMS (FAB) for C₂₄H₃₀N₁₀O₆S, 587(MH+), calcd 587.64.

Production of D1 Targeting Molecules

D1-apoE

The apoE peptide (200 μL, 2 mM) was slowly added (50 μL aliquot) to CAR D1 (300 μL, 0.24 mM) and 2 μL of 1M NaOH. After a 18 h incubation at 4° C., the pH of the solution was adjusted to 9 to 9.5 by addition of 8 μL of 1M NaOH. After a 48 h incubation at 4° C., the ligation reaction went essentially to completion as determined by SDS PAGE. The product was purified by size exclusion chromatography on a Superdex 75 HR 10/30 column (Amersham Pharmacia Biotech, Piscataway, N.J., 0.5 ml/min) using 10 mM Pipes pH 6.0, 100 mM NaCl as the elution buffer. The ligation mixture was injected into the column (500 μl injection loop) and collected fractions were analyzed by SDS PAGE. Fractions containing D1-apoE were combined and characterized by electrospray high mass resolution spectroscopy. HRMS (ESIMS) for C₉₂₃H₁₅₀₀N₂₆₄O₂₉₉S₆, 22,994.3, calcd 21,272.0 (M).

D1-FGF2

The thioester tagged D1 prepared as described above in Buffer A [20 mM Tris-HCl (pH8), 500 mM NaCl, 1 mM EDTA] was ligated to FGF2 by protein-protein ligation.¹⁰ CAR D1 (1 ml, 0.122 mM in Buffer A) was combined with FGF2 (1 ml, 0.127 mM in Buffer D) in a Centricon 3 centrifugal filter device and the ligation mixture was concentrated to a final volume of 460 μl (0.27 mM each protein). After a 48 hour incubation at 4° C., the product was purified by size exclusion chromatography as described above using 20 mM Tris-HCl (pH 7.5), 500 mM NaCl, 1 mM EDTA as the elution buffer and a 200 μl injection loop. Fractions containing homogenous D1-FGF2 were combined to yield approximately 800 μg of protein. HRMS (ESI) for C₁₅₀₆H₂₃₈₃N₄₂₀O₄₅₈S₁₂, 34,085.59 (MH+), calcd 34,084.7 (M).

D1-folate

L-cysteine-ethylenediamine-α,γ-folate (1.8 mg, 2.21 μmol) was resuspended in 100 μL Buffer E and dissolved with 6.6 μL of 1M NaOH. CAR D1 (366 μL, 0.59 mM) was combined with L-cysteine-ethylenediamine-α,γ-folate (80 μL, 20.7 mM) and the pH of the solution was adjusted to 8.5 to 9.0 by addition of 6 μL of 1M NaOH. After a 48 hour incubation at 4° C., the product was purified by size exclusion chromatography as described above using Buffer E as the elution buffer. Fractions containing D1-folate that were estimated to be >90% homogenous as determined by SDS PAGE were combined and incubated for 60 min with 750 μl of chitin beads to remove the cleaved intein that had eluted during the D1-GyrA cleavage step. Approximately 330 μg of homogenous D1-folate was isolated using this procedure. HRMS (ESIMS) for C₇₅₇H₁₁₉₄N₂₀₈O₂₄₃S₆, 17,430.3 (M+MESNA), calcd 17,289.59 (M). The observed mass corresponded to disulfide formation of D1-folate with MESNA at the ligation site. DTT can be used after the ligation step in order to reduce any unwanted disulfide bonds,¹¹ but was not used here to avoid reduction of the CAR D1 endogenous disulfide bond.

Adenoviral Transduction Assay

Cell Culture

The ovarian adenocarcinoma SKOV-3 cells were obtained from the American Type Culture Collection (ATCC HTB-77) and maintained in McCoy's 5A culture media containing 10% fetal bovine serum (FBS). Human fibroblast HDF cells were maintained in DMEM medium containing 10% FBS. KB cells (ATCC CCL-17) were maintained in folate-free RPMI 1640 medium containing 2% FBS.

Adenoviral Vectors

The adenoviral vector Av3GFP was used in these studies to evaluate the function of each adenoviral targeting molecule. Av3GFP, a third generation vector in which the E1, E2a, and E3 regions were deleted,¹² contains the green fluorescent protein (GFP) cDNA under the control of the RSV promoter. Viral particle titers (particles per ml) were determined as described previously.¹³

Adenoviral Transduction and Competition

Cells were seeded into 6-well plates at a density that achieved approximately 80% confluency after an overnight incubation. Av3GFP was incubated for 30 minutes at room temperature with the CAR D1 targeting molecules in 100 μl of serum free media. Following incubation, the complexes were diluted to 500 μl with culture medium containing 2% FBS and added to the cell monolayers that had been washed with PBS.

Cell transduction proceeded for 1 hour at 37° C. in a 5% CO₂ incubator, after which the cells were washed with PBS and further incubated 24 to 36 hours in 3 ml of culture media. Cells were harvested with Trypsin-Versene (Life Technologies, Inc.), detached with a cell-lifter and pelleted by centrifugation in 5 ml polystyrene tubes (12×75 mm, Falcon 2052). After fixing the cells in 300 μl to 2 ml PBS containing 1% paraformaldehyde, adenoviral-mediated GFP expression was analyzed by flow cytometry. Data are presented as the percentage of fluorescent positive cells (corrected for background controls). The data are the averages of duplicate determinations. Each experiment was repeated a minimum of three times. To demonstrate the specificity of complex formation or receptor interaction, the virus complexes were incubated with the appropriate competitor. For disruption of the viral complex, excess free recombinant Ad5 fiber knob protein was added. The DNA fragment encoding the Ad5 fiber knob domain was cloned into pET15b24 and recombinant Ad5 fiber knob was purified as described previously.¹⁴ The competitor used to assess the interaction of the synthetic ligand, folate, with its receptor was excess free folic acid. The competitors used to assess the interaction of the apoE peptide with its receptor were a mouse monoclonal antibody against the human LDL receptor-related protein (LRP) (Research Diagnostics, Inc., Flanders, N.J.) or the free apoE peptide. The competitors used for the FGF2 constructs included recombinant FGF2 or a rabbit anti-FGF2 polyclonal antibody (Sigma Chemical Co., St. Louis, Mo.).

Results

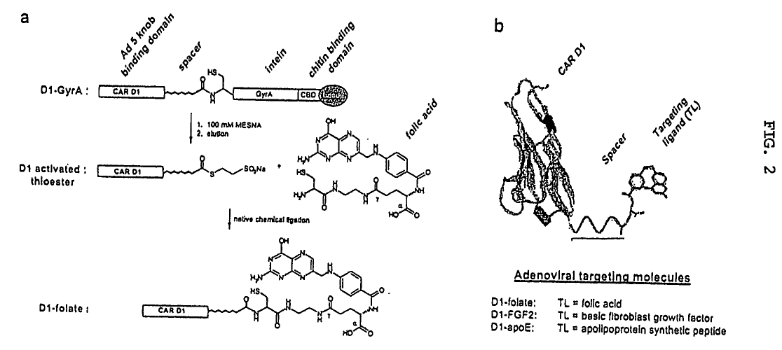

FIG. 2 shows the synthesis of adenoviral targeting molecules. FIG. 2a represents intein mediated protein ligation of folic acid to CAR D1. (Abbreviations: GyrA, mycobacterium xenopi GyrA; CBD, chitin binding domain). The spacer amino-acid sequence is SSSNNNNNNNNNNLGIEGRGTLEM (SEQ ID NO:3). The intein catalyzes a slow N-S acyl shift of the spacer carboxy terminus to the sulfhydryl atom of the GyrA N-terminal cysteine thereby providing a reactive thioester intermediate at the junction of D1-GyrA, which is cleaved by an excess of 2-mercaptoethanesulfonic acid (MESNA).¹⁵ The cleavage reaction releases CAR D1 with a C-terminal activated thioester while GyrA-CBD remains bound on the chitin column. FIG. 2b represents a structural representation of the Ad targeting molecule D1-folate. Targeting ligands (TL) are attached to the CAR D1 moiety (ribbon structure) bearing a spacer at its C-terminus to generate D1-folate, D1-FGF2, and D1-apoE.

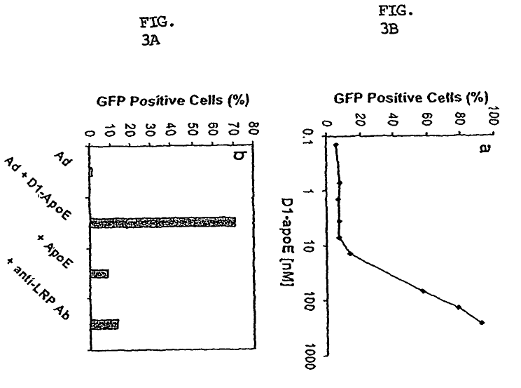

FIGS. 3A and 3B show the LRP-mediated Ad transduction of HDF cells. In FIG. 3A, Av3GFP, 5000 particles per cell, was mixed with increasing concentrations of D1-apoE from 0.14 to 280 nM. After 24 hours, the adenoviral-mediated expression of GFP was analyzed as described. FIG. 3B shows the results of a competition assay. HDF cells were transduced with 5000 ppc Av3GFP premixed with 470 nM D1-apoE as described. 20 μM apoE peptide or 10 μg rabbit anti-LRP polyclonal antibody were prebound for 30 min at 37° C. on the cell monolayer, prior to the addition of the D1-apoE bound complex. These results demonstrate that ligation of peptides to D1 via intein mediated ligation produces functional molecules that can retarget adenovirus to alternative receptors.

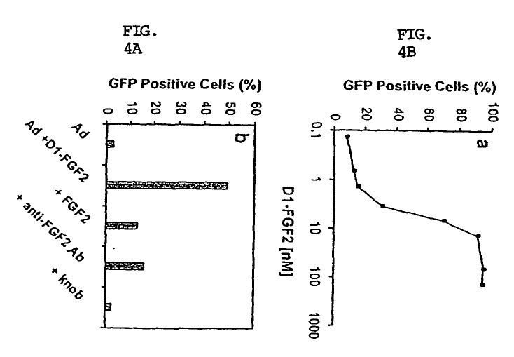

FIGS. 4A and 4B represent FGFRs-mediated transduction of SKOV-3 cells. In FIG. 4A, Av3GFP, 200 particles per cell, was mixed with increasing concentrations of D1-FGF2 from 0.14 to 140 nM. GFP expression was measured as described. FIG. 4B shows the results of a competition assay. SKOV-3 cells were transduced with 200 ppc of AV3GFP premixed with 6 nM D1-FGF2. Competition experiments were carried out in the presence of one of the following competitors: 20 μg FGF2, 8.8 μg of rabbit anti-FGF2 polyclonal antibody or 10 μg fiber knob, prior to addition to the cells. These results demonstrate that proteins bearing a N-terminal cysteine can be successfully ligated to D1 using intein mediated ligation and used as adenoviral targeting ligands.

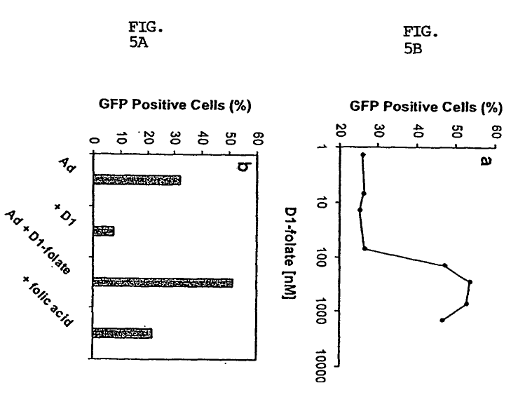

FIGS. 5A and 5B show FRs-mediated Ad transduction of KB cells. In FIG. 5A, Av3GFP, 100 particles per cell, was mixed with increasing concentrations of D1-folate from 1.4 to 1400 nM. GFP expression was measured as described above. FIG. 5B shows the results of a competition assay. KB cells were transduced with 100 ppc Av3GFP premixed with 718 nM D1-folate or 25 μg D1 as described. Competition experiments were carried out in the presence 50 μg folic acid, prior to addition to the cells. CAR D1 was prepared as described for D1-apoE except 5 mM cysteine was used during the ligation step. These results demonstrate that small molecules can be successfully ligated to D1 by intein mediated protein ligation and used as reagents to retarget adenovirus to alternative receptors.

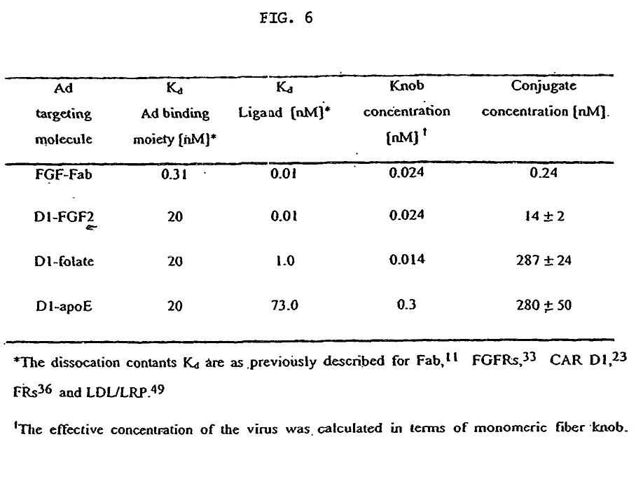

FIG. 6 represents the correlation between binding affinity and amount of CAR D1 targeting molecules required for optimal targeted gene delivery. The amount of each D1-targeting molecule that was needed for maximum transduction was compared relative to the affinity of D1 interacting with fiber knob or each ligand with its receptor. D1-FGF2 was compared to FGF-Fab, a bifunctional target conjugate known to retarget Ad to FGFRs through binding of the same targeting ligand FGF2¹⁶ in SKOV3 cells. As shown in Table 2, 14 nM of D1-FGF2 was required to achieve maximal GFP expression as opposed to only 0.24 nM FGF-Fab as previously reported.¹⁷ These results indicate that the use of a higher affinity adenovirus-binding moiety such as the anti-fiber Fab achieves a stronger association with the adenoviral capsid that results in the use of lower concentrations of the Ad targeting molecule.

While the invention has been described in detail and with reference to specific examples thereof, it will be apparent to one skilled in the art that various changes and modifications can be made therein without departing from the spirit and scope thereof.

What is claimed is:

1. A composition comprising a surface and a modified protein, and optionally a gene transfer vector, wherein the gene transfer vector is bound to the modified protein and the modified protein is covalently bound to the surface.

2. The composition of claim 1, wherein the gene transfer vector is adapted to bind to a receptor on the mammalian cell and wherein the modified protein comprises at least one of a fusion protein and a polypeptide.

3. The composition of claim 1, wherein the modified protein is covalently bound to the surface through a thiol residue and a linker.

4. The composition of claim 1, wherein the gene transfer vector is a viral vector.

5. The composition of claim 4, wherein the viral vector is an adenovirus vector.

6. The composition of claim 5, wherein the adenovirus vector is a member selected from the group consisting of a first-generation adenovirus vector, a second-generation adenovirus vector, an adenovirus vector of large DNA capacity and a deleted adenovirus vector.

7. The composition of claim 1, wherein the surface is a metal surface.

8. The composition of claim 7, wherein the metal surface is a surface of a medical device.

9. The composition of claim 8, wherein the medical device is selected from the group consisting of a stent, a heart valve, a wire suture, a joint replacement, a urinary dilator, an orthopedic dilator, a catheter and a endotracheal tube.

10. The composition of claim 8, wherein the medical device is at least one of an internal device and an external device.

11. The composition of claim 8, wherein the medical device is coated with a layer of the linker, a layer of the modified protein and a layer of the gene transfer vector.

12. The composition of claim 2, wherein the fusion protein is generated through intein-mediated protein ligation.

13. The composition of claim 2, wherein the fusion protein comprises at least a fragment of a CAR protein and a receptor targeting ligand.

14. The composition of claim 13, wherein the fragment of the CAR protein is an extracellular domain of CAR or an immunoglobulin D1 domain of CAR.

15. The composition of claim 13, wherein the receptor targeting ligand is selected from the group consisting of apolipoprotein E, transferrin, a vascular endothelial growth factor, a transforming growth factor-beta, a fibroblast growth factor, an RGD containing peptide and folic acid.

16. The composition of claim 2, wherein the receptor is selected from the group consisting of a lipoprotein receptor, a transferrin receptor, a VEGF receptor, a TGF-beta receptor, an FGF receptor, a recombinant integrin receptor protein, a folic acid receptor and a folate receptor.

17. A method for preparing the composition of claim 1, the method comprising:
(a) providing a protein;
(b) modifying the protein with a reagent to contain a reactive group, thereby yielding a modified protein;
(c) providing a surface;
(d) treating the surface with a surface modifier comprising a linker and a functional group;
(e) reacting the modified protein with the functional group on the surface in order to covalently bind the modified protein to the surface via the linker; and optionally
(f) binding the gene transfer vector to the modified protein.

18. The method of claim 17, wherein the protein is a CAR protein or fragment of CAR.

19. The method of claim 18, wherein the fragment of CAR is an immunoglobulin D1 domain of CAR.

20. The method of claim 17, wherein the protein is a fusion protein.

21. The method of claim 20, wherein the fusion protein comprises a fragment of CAR ligated to a receptor targeting ligand by intein-mediated protein ligation.

22. The method of claim 21, wherein the fragment of CAR is an extracellular domain of CAR or an immunoglobulin D1 domain of CAR.

23. The method of claim 21, wherein the receptor targeting ligand is selected from the group consisting of apolipoprotein E, transferrin, a vascular endothelial growth factor, a transforming growth factor-beta, a fibroblast growth factor, an RGD containing peptide and folic acid.

24. The method of claim 17, wherein the reagent is a cysteine and the reactive group is a thiol group or an avidin-biotin affinity construct.

25. The method of claim 17, wherein the surface is a surface of a medical device.

26. The method of claim 25, wherein the medical device is selected from the group consisting of a stent, a heart valve, a wire suture, a joint replacement, a urinary dilator, an orthopedic dilator, a catheter and a endotracheal tube.

27. The method of claim 25, wherein the medical device is at least one of an internal device and an external device.

28. The method of claim 17, wherein the surface modifier is polyallylamine bisphosphonate, the linker is an entity containing a reactive succinimide and a pyridyl-dithiol group, and the functional group is selected from the group consisting of an amino group, a sulfhydryl group, biotin reactive succinimides, epoxy-residues and aldehyde functionalities.

29. The method of claim 17, wherein the gene transfer vector is a viral vector.

30. The method of claim 29, wherein the viral vector is an adenovirus vector.

31. The method of claim 30, wherein the adenovirus vector is a member selected from the group consisting of first-generation adenovirus vector, second-generation adenovirus vector, adenovirus vector of large DNA capacity and deleted adenovirus vector.

32. A method of delivering a viral vector to an animal tissue, the method comprising administering to a body location in fluid communication with the animal tissue the composition of claim 1.


SEQUENCE LISTING (3 sequences)

SEQ ID NO: 1 - protein, 38 aa, Artificial
  apoE targeting peptide
CLRKLRKRLLRDADDLLRKLRKRLLRDADDLGSDDDDD

SEQ ID NO: 2 - DNA, 31 bp, Artificial
  Primer Sequence
cggggtccgggagaaaagcgaccctcacatc

SEQ ID NO: 3 - protein, 24 aa, Artificial
  Spacer Sequence
SSSNNNNNNNNNNLGIEGRGTLEM